(19) United States
(12) Patent Application Publication
(10) Pub. No.: US 2025/0194922 A1
(43) Pub. Date: Jun. 19, 2025

(54) OBSERVATION DEVICE AND METHOD FOR OBSERVING AN EYE

(71) Applicant: Oculus Optikgeraete GmbH, Wetzlar (DE)

(72) Inventors: Oliver Schepp, Lich (DE); Lars Risby, Nyborg (DK); Thomas Rompf, Lahnau (DE)

(21) Appl. No.: 18/983,292

(22) Filed: Dec. 16, 2024

(30) Foreign Application Priority Data
Dec. 15, 2023 (DE) 10 2023 135 378.0

(51) Int. Cl. A61B 3/13

(52) U.S. Cl. CPC A61B 3/13

(57) ABSTRACT

An observation device having a positioning unit for positioning an optical unit in a beam path of a microscope objective and an eye to be observed, and a related method for observing an eye. The positioning unit includes a connection device, a positioning device, an accommodating device and the optical unit having a lens for observing a fundus of the eye, and a further optical element, the positioning unit comprising a pivoting mechanism by which the optical unit is pivotable out of or into the beam path, the positioning unit being couplable to the microscope by the connection device, the lens being adapted on the positioning device by the accommodating device, the accommodating device at least predominantly made of plastic material, wherein the positioning device has a lens barrel which is disposed on the pivoting mechanism in a pivotable manner, the lens barrel at least predominantly made of metal.

This application claims priority to German Patent Application No. 10 2023 135 378.0 filed on Dec. 15, 2023, the disclosure of which is hereby incorporated by reference.

### TECHNICAL FIELD

The invention relates to an observation device having a positioning unit for positioning an optical unit in a beam path of a microscope between an objective of the microscope and before an eye to be observed, the positioning unit comprising a connection device, a positioning device, an accommodating device and the optical unit, the optical unit comprising a lens, which serves to observe a fundus of the eye, and a further optical element, the positioning unit comprising a pivoting mechanism by means of which the optical unit is pivotable out of or into the beam path, the positioning unit being couplable to the microscope by means of the connection device, the lens being adapted on the positioning device by means of the accommodating device. Furthermore, the invention relates to a method for observing an eye with an observation device of this kind, the accommodating device being at least predominantly, preferably completely, made of plastic material, Furthermore, the invention relates to a method for observing an eye with an observation device of this kind.

### BACKGROUND

Microscopes for performing eye surgery are typically used for surgeries in an anterior region of an eye. If interventions of this kind are also to be performed in a posterior region of the eye, it is necessary to supplement the microscope with an observation device that enables a focusing of exactly this region of the eye. Observation devices of this kind comprise at least one wide-angle lens and/or ophthalmoscopy lens for the wide-angle viewing of the respective posterior part of the eye, wherein the ophthalmoscopy lens provides an intermediate image in a beam path before an objective of the microscope, wherein said intermediate image can be focused with the microscope. To focus the intermediate image, a shortening of the length of the beam path of the microscope is needed, which can be carried out by means of the corresponding setting mechanism on the microscope. However, since it is necessary to switch between different views, with and without an ophthalmoscopy lens, during an eye surgery, such a setting of the microscope is hindering, so that a so-called reduction lens can be provided in the beam path before the objective, wherein said reduction lens serves to shorten the beam path of the microscope and is used together with the ophthalmoscopy lens. The two lenses are held as an optical unit by a positioning unit of the observation device, which is fixed directly to the microscope, and can be positioned in the beam path as necessary without the microscope needing to be substantially adjusted during a surgery. The positioning unit typically comprises a connection device by means of which the positioning unit is couplable to the microscope. Furthermore, the positioning unit is designed in such a manner that the respective lenses can simply be swung into or inserted into the beam path and removed therefrom again.

An observation device of this kind is known from DE 10 2011 002 940 Al, for example. In order to be able to adjust the intermediate image of the ophthalmoscopy lens as precisely as possible to the focal length of the objective of the microscope, the ophthalmoscopy lens is designed to be settable along the beam path of the microscope by means of a screw drive.

This observation device is realized in such a manner that the ophthalmoscopy lens can be moved relative to the eye along the beam path. In this case it is advantageous if the ophthalmoscopy lens is as close to the eye as possible since, in this case, a comparatively large area of the eye is clearly visible. However, at the same time, it has to be avoided that the ophthalmoscopy lens comes into contact with the eye. In order to obtain a sharp image of an area of the eye which is as large as possible, it is, therefore, always required to vary a distance of the microscope relative to the eye and to coordinate it with the distance of the ophthalmoscopy lens. If a different distance of the ophthalmoscopy lens to the eye is needed for a certain procedure step during an eye surgery, for example if the aqueous humor of the eye is to be aspirated, this adjustment of the respective relative distances from the ophthalmoscopy lens to the microscope and to the eye and the focusing of the image then obtained have to be carried out again.

Nowadays, it is considered advantageous to be able to dispense with a sterilization of the observation device and/or positioning unit if it is/they are made of plastic and can be used as a disposable product. Thus, observation devices which are made of plastic and enable a one-time use of the observation device are known. DE 10 2018 127 469 B4 shows such an observation device.

However, in this case it is disadvantageous that plastic, unlike metal, cannot always be used in the desired precision required for the ophthalmoscopy lens to be positioned, in particular if the positioning device or the positioning unit is made of fragile plastic bars. Thus, a displacement of the ophthalmoscopy lens along the beam path or an offset transverse thereto can easily occur when the ophthalmoscopy lens is swung into or out of the beam path. Typically, this then requires a correction of the position of the ophthalmoscopy lens, which is hindering for the performance of an eye surgery.

### SUMMARY

The object of the invention at hand is therefore to propose an observation device and a method for observing an eye, which enable an improved handling during an eye surgery.

In the observation device according to the invention having a positioning unit for positioning an optical unit in a beam path of a microscope between an objective of the microscope and before an eye to be observed, the positioning unit comprises a connection device, a positioning device, an accommodating device and the optical unit, the optical unit comprising a lens, which serves to observe a fundus of the eye, and a further optical element, the positioning unit comprising a pivoting mechanism by means of which the optical unit is pivotable out of or into the beam path, the positioning unit being couplable to the microscope by means of the connection device, the lens being adapted on the positioning unit by means of the accommodating device, the accommodating device being at least predominantly, preferably completely, made of plastic material, wherein the positioning device has a lens barrel which is disposed on the pivoting mechanism in a pivotable manner, the lens barrel being at least predominantly or completely made of metal.

The observation device according to the invention can be adapted to a microscope and/or detachably connected to it by means of the connection device. In this case, the lens, which can be an ophthalmoscopy lens, is held into the beam path of the objective between the eye to be observed and the objective by means of the positioning unit. In this case, the intention is for the lens to be disposed in such a manner that a main axis and/or an optical axis of the objective of the microscope run(s) through a central point of the lens. By means of the pivoting mechanism, the optical unit having the lens and the further optical element, which is preferably a lens having positive refractive power, can be swung into the beam path or swung out again as necessary during an eye surgery. In this case it is initially irrelevant how the pivoting mechanism is designed; what is essential is that the optical unit can be completely removed from the beam path and moved into it. The pivoting mechanism can therefore also be seen as a displacement mechanism by means of which the optical unit can be displaced parallel to the beam path.

Furthermore it is advantageous that the accommodating device holding the lens is essentially made of plastic material and the lens barrel is essentially made of metal. Thus, the pivotable lens barrel can be designed in a particularly stable and precise manner and allows a precise positioning of the lens and the further optical element in the beam path without it being necessary, in this case, to carry out a corrective adjustment, as is the case with pure disposable products. At the same time, it is possible to produce the accommodating device with the lens particularly cost-efficiently due to the fact that the accommodating device consists of plastic. The production can be carried out easily and in large quantities, for example in the context of an injection molding process. This, in turn, makes it possible to use the accommodating device as a disposable product. In this case, the accommodating device can be disposed of after an eye surgery has been performed. A sterilization of the accommodating device is not necessary. For the performance of a subsequent eye surgery, a new accommodating device which can be packaged sterilely can be used. The accommodating device can be easily adapted on the lens barrel and/or detachably connected to it.

The further optical element can be disposed below the pivoting mechanism. Since the positioning device has a lens barrel which is disposed on the pivoting mechanism in a pivotable manner, an arrangement of the further optical element in the lens barrel is enabled. Thus, a movable arrangement of the further optical element within the lens barrel is particularly easily possible. In this case, the further optical element can also be easily protected from external influences.

The further optical element can be movable in the longitudinal direction of the beam path relative to the microscope by means of the positioning device. In this case, the movable arrangement of the further optical element below the pivoting mechanism makes it possible to adjust the beam path of the microscope with the further optical element and/or to shorten this beam path to such an extent that an intermediate image of the lens can be focused. If the further optical element can be moved along the beam path below the pivoting mechanism, comparatively more space is available for moving the further optical element along the beam path than if the further optical element was disposed above the pivoting mechanism. In this case, a distance between the pivoting mechanism and the objective of the microscope is comparatively short, as this is the only way to be able to ensure complete removal of the optical unit and/or the positioning device from the beam path. The comparatively larger adjustment range of the further optical element makes it possible to adjust the observation device universally to different microscope types and thus to use it for these. In this case, it is no longer necessary to design the further optical element individually for different microscopes with different beam paths. In addition, the lens can also remain fixed in a position in the beam path and does not have to be moved relative to the microscope along the beam path. It is only necessary to align the microscope together with the lens with the eye. In this case, the adjustment of the beam path can be carried out simply by moving the further optical element. The lens and the further optical element can each be composed of a plurality of optical components which are connected to each other and together form an optical element in each case. The lens can be an ophthalmoscopy lens, the further optical element being at least one lens having positive refractive power and serving to adjust the beam path, the lens in the lens barrel being disposed movably in the longitudinal direction of the beam path below the pivoting mechanism. The lens having positive refractive power can be a so-called reduction lens by means of which the beam path of the microscope can be shortened. Since the lens having positive refractive power is movable along the beam path in the lens barrel below the pivoting mechanism, the adjustment of the beam path can be carried out easily with a comparatively large adjustment range. The lens having positive refractive power can be displaced along the beam path simply by means of a screw drive, a helix formed inside the lens barrel or the like. In this case, the lens barrel may be rotated at least partially. In this case, the lens having positive refractive power can be accommodated in a mount which is displaceable inside the lens barrel together with the lens.

The observation device can comprise a cover unit made of plastic material for sterilely covering the pivotable lens barrel of the positioning device. The cover unit can be realized in a comparatively thin-walled manner so that the cover unit can be in close contact with the pivotable lens barrel. The plastic material can be a rigid or a flexible plastic material. Furthermore, the plastic material can be opaque or optically partially transparent. In particular, the cover unit can be constituted in such a manner that the pivotable lens barrel is completely shielded on its outer surface from an environment by the cover unit. In this case, the pivotable lens barrel can be manually grasped and actuated by a surgeon even without the need for a subsequent sterilization of the lens barrel. In this case, it is only necessary to remove the cover unit which can be manufactured cost-efficiently from plastic material and to replace it with a new, sterile cover unit which has not been used up to now.

The cover unit can have an upper sterile cover for at least partially covering an end surface of the lens barrel and a lower sterile cover for at least partially covering a circumferential surface of the lens barrel. Accordingly, the cover unit can be realized in two parts. The upper sterile cover can be placed on the end surface of the lens barrel from above when the lens barrel is pivoted out of the beam path. The lower sterile cover can be placed on the lens barrel from below. In this case, the lens barrel is surrounded on all sides by the cover unit. In particular the upper sterile cover allows a protection of the end surface of the lens barrel from a touch of the surgeon when he/she manually grasps the lens barrel in a position pivoted out of the beam path. If an upper sterile cover is used, it is also advantageous if a gap within which the upper sterile cover can be located when the lens barrel is swung into the beam path is realized between the lens barrel and the connection device.

The cover unit can be realized at least with one connecting element which requires a destruction of the connecting element when separating the cover unit from the positioning device. The connecting element can be realized in the manner of a latching element which engages a projection realized on the lens barrel or engages into a groove realized on the lens barrel. It may also be provided a plurality of connecting elements. The connecting element can be flexibly realized or mounted so that the connecting element can easily come into contact with the lens barrel. It is essential that the connecting element is constituted in such a manner that the connecting element and/or the cover unit are/is destroyed when the cover unit is removed from the positioning device and/or the lens barrel. This prevents the cover unit from being used again by mistake.

The cover unit can have at least one tear strip by means of which the cover unit is at least partially destroyable. The tear strip can be realized having a tab which can be manually easily grasped. The tear strip can be realized by a line of weakness or two parallel lines of weakness in the cover unit. If the cover unit is fixed to the positioning device and/or the lens barrel, for example, by means of a latching connection, the latching connection can be destroyed by manually actuating the tear strip. This makes it possible to easily remove the cover unit from the positioning device and/or the lens barrel.

The accommodating device can be realized with at least one connecting element which requires a destruction of the connecting element when separating the accommodating device from the positioning device. This can also prevent the accommodating device from being reused by mistake after the separation from the positioning device. The connecting element can be realized, for example, in such a manner that the connecting element breaks when the accommodating device is removed from the positioning device.

The accommodating device can be realized having a further, preferably conical, lens barrel. The further lens barrel can be directly attached to the lens barrel and fixedly connected thereto. The fixed connection can be realized by a latching connection, for example. It is particularly advantageous if the further lens barrel is realized in the manner of a cone. In this case, the lens barrel can be adjusted to a shape of a beam path in such a manner that a diameter of the further lens barrel is comparatively small at a lower end of the further lens barrel. In this case, the lens can be disposed on the lower end. It is also advantageous if the further lens barrel is closed. In this case, the further lens barrel can be realized in the manner of a conical sleeve.

The further lens barrel can be composed of an upper section and of a lower section, wherein the lower section can be mounted loosely or in a spring-loaded manner on the upper section in such a manner that the lower section is insertable into the upper section. The spring-loaded mounting can be realized by means of a compression spring which is inserted into the upper section and against the spring force of which the lower section can be moved into the upper section. This can prevent the eye from being injured in an undesirable way in case of a possible collision of the lens and/or the lower section with an eye of a person to be operated on.

At least one manually actuable projection can be realized on the lower section, wherein the projection can pass through a longitudinal slot, which is realized in the upper section, and be movable along the longitudinal slot. In this case, a surgeon can manually move the lower section in the direction of the beam path in that the surgeon grasps the projection and pulls it upward in the direction of the microscope in such a manner that the lower section is moved into the upper section. Advantageously, for this purpose, two opposing projections can also be realized on the lower section, said projections each engaging into longitudinal slots, which oppose each other, on the upper section. A surgeon can advantageously use the facility of manually moving the lower section in the direction of the beam path away from the eye when the optical unit is to be pivoted out of the beam path. In particular, if the lens is particularly close to the eye to be operated on, the lens can be manually moved out of a danger zone for the eye and the optical unit can be pivoted out of the beam path in an immediately subsequent manual movement. The same applies to a reverse movement of the optical unit into the beam path. In this case, a corresponding movement of the microscope is no longer required.

The positioning device can have a drive unit by means of which a position of the further optical element is settable in the longitudinal direction of the beam path. The drive unit can be operated purely manually or also electrically. It is essential that the further optical element can be displaced and positioned along the beam path by means of the drive unit. In this respect, it is also advantageous if the drive unit is self-locking. If the further optical element is disposed within the lens barrel, the drive unit can also be realized at least partially or completely on the lens barrel.

The drive unit can be disposed on the connection device above the pivoting mechanism and/or below the pivoting mechanism on the lens barrel. Accordingly, the drive unit can be disposed solely on the lens barrel or also be realized in such a manner that the drive unit is disposed on the connection device and the lens barrel. Depending on the design of the drive unit, it can be advantageous to only realize a part of the drive unit on the lens barrel so that a surgeon is not hindered in his/her work by protruding components of the observation device.

The observation device can comprise a shielding unit for shielding an optical path of the positioning unit, wherein the shielding unit can be composed of at least one optically shielded or closed lens barrel. The positioning device and the accommodating device can realize this closed lens barrel, for example. Advantageously, it can thus be avoided that light sources used during an eye surgery, scattered light or the like enter the beam path and influence a representation of the image of the eye, which is observed by a surgeon through the optical unit, in an undesirable manner. Possible differences in brightness, reflections or the like can thus be avoided.

The drive unit can comprise a step motor which can be coupled to a coupling of the drive unit to the lens barrel via a belt drive or a transmission. In this case, the step motor can be an electric motor with which a defined number of revolutions can be carried out until the further optical element is at the desired position of the lens barrel. For this purpose, the lens barrel can be realized to be rotatable in sections so that revolutions of the step motor can be transferred to the lens barrel via the belt drive and/or the transmission. The drive unit can comprise, for example, a sleeve within the lens barrel, said sleeve being realized with a thread or a helix and being connected to the belt drive and/or transmission via the coupling. In this case, a rotation of the sleeve, which can be moved by means of the step motor, can carry out a lifting or lowering of the further optical element and/or a movement of the same along the beam path.

The coupling is separable or connectable by means of the pivoting mechanism. This is in particular then advantageous if the step motor having the belt drive or the transmission is disposed above the pivoting mechanism on the connection device. In this case, the coupling can be realized in such a manner between the connection device and the lens barrel that the coupling is separated when the lens barrel is pivoted out of the beam path and that the coupling is connected when the lens barrel is pivoted into the beam path. The coupling can be realized as a nonpositive, form-fitting and/or frictional coupling.

It is advantageous if the coupling is a magnetic coupling composed of two rings which are able to transfer a torque by means of magnets, wherein the further optical element can be movable by means of a rotation of the lens barrel. The two coaxial rings can each have a number of magnets which exert a magnetic force in such a manner on each other that the rings attract each other and, thus, a torque can be transferred. The magnets can be disposed at a regular distance on an axial end face of the respective rings. The poles of the magnets of the respective rings can alternate so that the rings are in a defined relative position when the coupling is closed. It is particularly advantageous if a gap is realized between the rings since, in this case, the rings and/or the coupling do/does not have to be in contact with each other to transmit the torque. The gap can be used to insert a sterile cover into the coupling and/or between the lens barrel and the connection device.

The positioning unit can comprise a control device, wherein the control unit can be realized to detect an optical unit pivoting out of or into the beam path and to transmit it to the microscope. The swinging in or out of the optical unit and/or the lens barrel can easily be detected by means of a sensor of the control device. In this case, the control device can signal to the microscope whether the optical unit is swung into or swung out of the beam path. If the microscope is equipped with a so-called inverter, the microscope can move the inverter into or out of the beam path within the microscope. In this case, a beam transposition and a mirror image of the intermediate image of the lens can be produced by means of the inverter so that a surgeon is presented with an image of the eye in correct positional arrangement when the optical unit is swung into the beam path.

The positioning unit can comprise a control device, a rotation of the lens barrel being detectable by means of a sensor of the control device, the drive unit being controllable by means of the control device in such a manner that the further optical element is movable in the longitudinal direction of the beam path to a presupposed position by means of the drive unit. The sensor can be, for example, a Hall sensor disposed on the drive unit and/or the lens barrel. A mark, a number of marks in the manner of a scale or the like, can be arranged on the lens barrel so that a rotation and position of the further optical element in the longitudinal direction of the beam path can be detected by means of the sensor. This then enables the detection of a position of the further optical element along an adjustment range. If, for example, an unintentional rotation of the lens barrel or a rotation of the connection device on the objective of the microscope occurs when the optical unit and/or the lens barrel are/is pivoted out of the beam path, the further optical element is no longer in the presupposed position and/or no longer in a focus, which was set by the surgeon before pivoting out, when it is pivoted into the beam path. By means of the drive unit, the control unit can now move the optical element to the presupposed position and/or the focus of the optical unit set before. In this case, it is no longer necessary for the surgeon to actuate the drive unit to correct a changed setting of the further optical element.

The microscope according to the invention comprises an observation device according to the invention. Further advantageous embodiments of the microscope are provided by the descriptions of features of the dependent claims referring back to claim 1.

In the method according to the invention for observing an eye with an observation device, an optical unit is positioned in a beam path of a microscope between an objective of the microscope and before an eye to be observed by means of a positioning unit of the observation device, the positioning unit comprising a connection device, a positioning device, an accommodating device and the optical unit, the optical unit comprising a lens, which serves to observe a fundus of the eye, and a further optical element, the positioning unit comprising a pivoting mechanism by means of which the optical unit is pivoted out of or into the beam path, the positioning unit being coupled to the microscope by means of the connection device, the lens being adapted on the positioning unit by means of the accommodating device, the accommodating device being at least predominantly, preferably completely, made of plastic material, wherein a lens barrel of the positioning device is pivoted, said lens barrel being disposed on the pivoting mechanism, the lens barrel being at least predominantly or completely made of metal. Regarding the advantages of the method according to the invention, reference is made to the description of advantages of the observation device according to the invention.

The further optical element can be used for the correction of refractive errors of the eye. Since the further optical element allows an intermediate image of the lens to be focused and, thus, the beam path of the microscope to be adjusted, a refractive error of the eye can also be corrected with the further optical element.

Further advantageous embodiments of the method are provided by the descriptions of features of the dependent claims.

### BRIEF DESCRIPTION OF THE DRAWINGS

Below, a preferred embodiment of the invention is explained in more detail with reference to the accompanying drawings.

In the figures:

FIG. 1 shows a perspective view of an observation device;

FIG. 2 shows a side view of the observation device with a shielding unit;

FIG. 3 shows a side view of the observation device without the shielding unit;

FIG. 4 shows a side view of the observation device with a positioning device swung out of a beam path;

FIG. 5 shows a longitudinal section view of the observation device with the shielding unit;

FIG. 6 shows a longitudinal section view of the observation device without the shielding unit;

FIG. 7 shows a perspective view of the observation device with the positioning device swung out of the beam path and the shielding unit;

FIG. 8 shows a perspective view of the observation device without the shielding unit and an accommodating device;

FIG. 9 shows a longitudinal section view of the shielding unit and the accommodating device;

FIG. 10 shows an exploded view of the shielding unit and the accommodating device.

### DETAILED DESCRIPTION

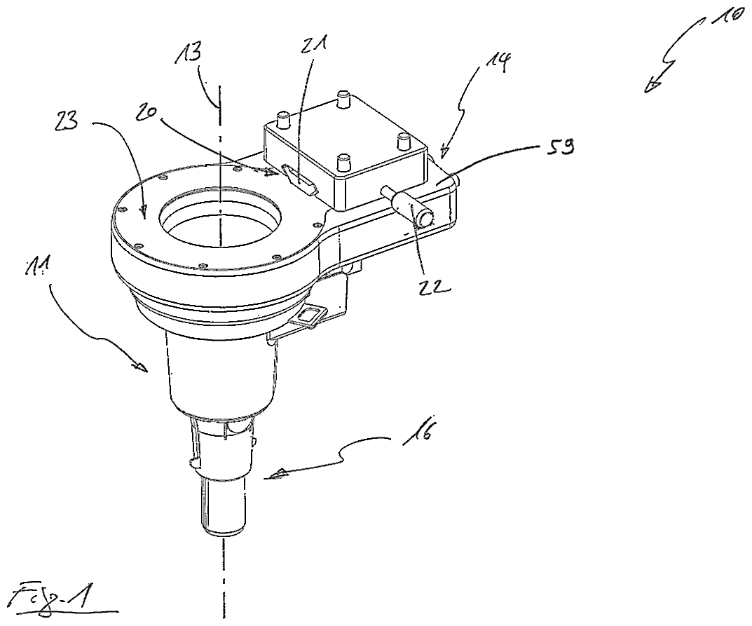

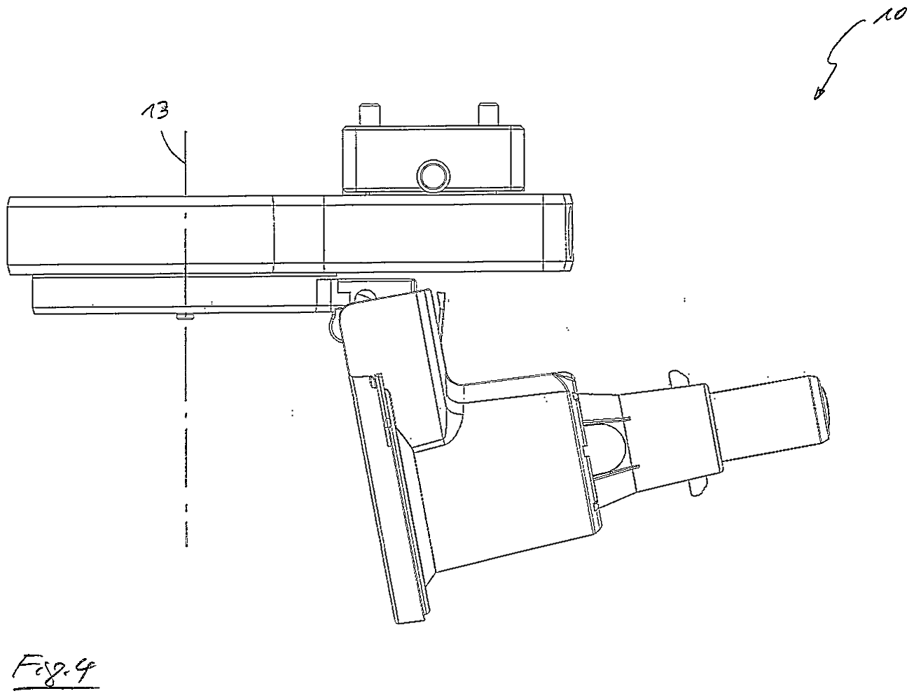

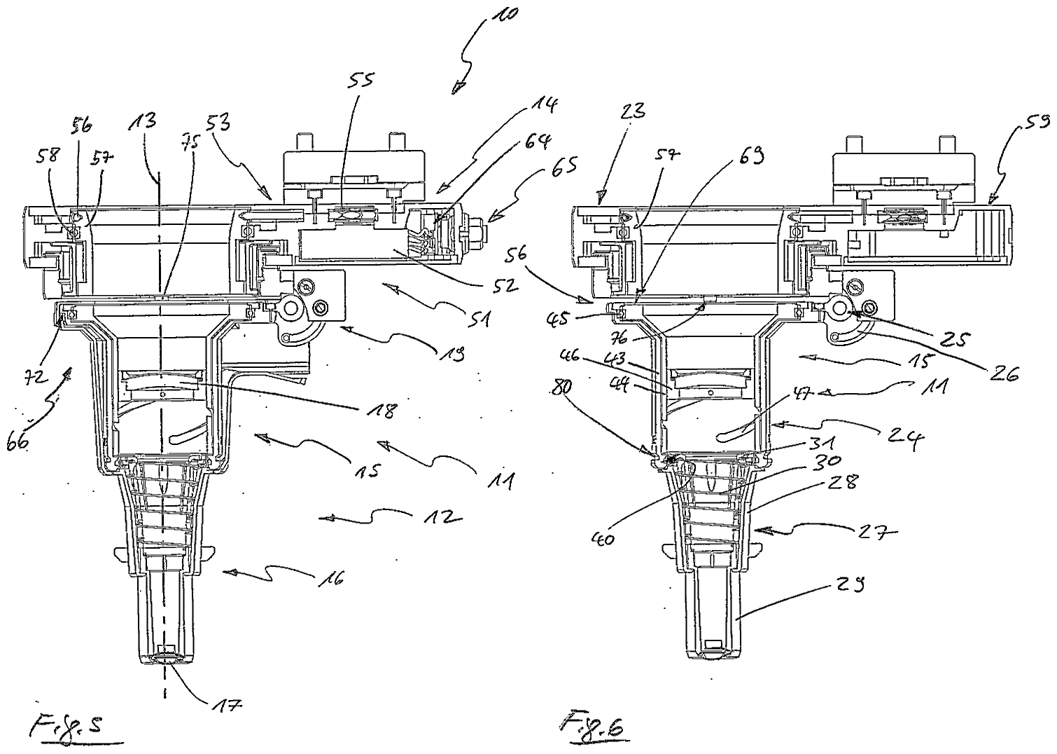

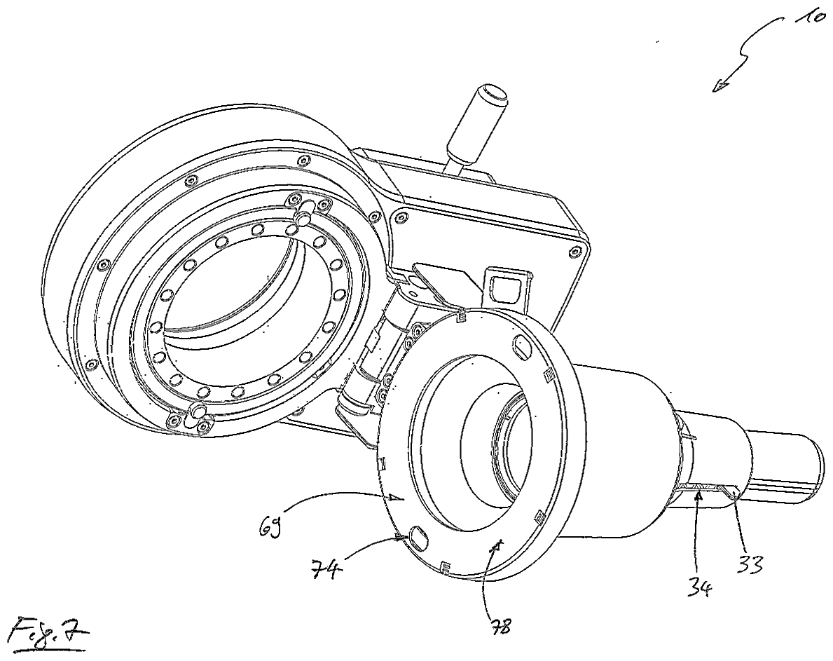

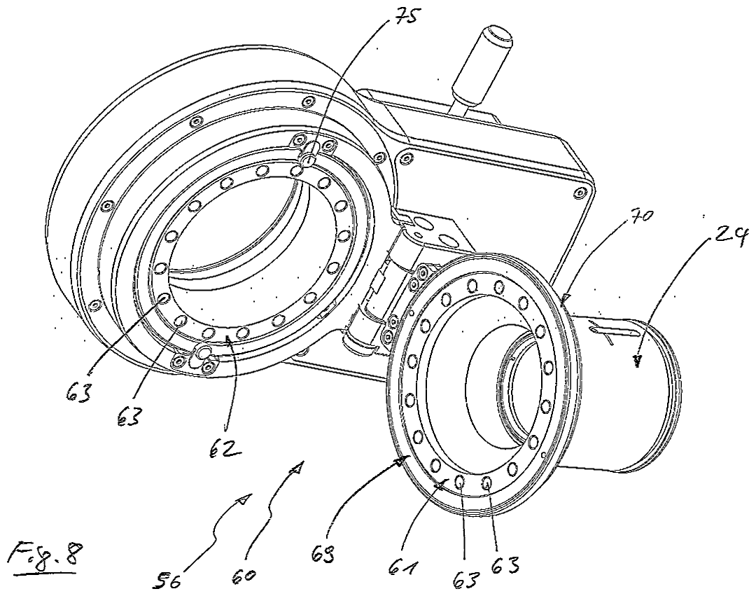

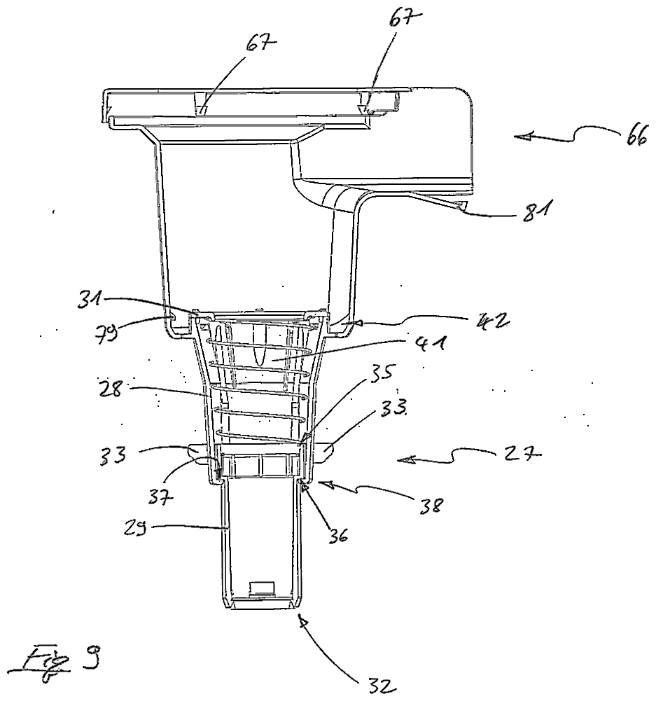

A combination of FIGS. 1 to 8 shows an observation device 10 having a positioning unit 11 for positioning an optical unit 12 in a beam path 13 of a microscope (not shown in more detail in the case at hand). Observation device 10 is adaptable on the microscope between an objective of the microscope and before an eye to be observed. Positioning unit 11 comprises a connection device 14, a positioning device 15, an accommodating device 16 and optical unit 12. Optical unit 12 is composed of an ophthalmoscopy lens 17 and a lens having positive refractive power or a reduction lens 18. In the case at hand, ophthalmoscopy lens 17 serves to observe a fundus of the eye and reduction lens 18 serves to adjust beam path 13 of the microscope to an intermediate image (not visible in the case at hand) of ophthalmoscopy lens 17. Furthermore, positioning unit 11 comprises a pivoting mechanism 19 by means of which optical unit 12 is pivotable into or out of beam path 13. FIGS. 1 to 3, 5 and 6 show positioning device 15, which is swung into beam path 13, with optical unit 12 and FIGS. 4, 7 and 8 show positioning device 15, which is swung out of beam path 13, with optical unit 12.

Furthermore, positioning unit 11 is couplable to a microscope by means of connection device 14. In the case at hand, connection device 14 is, inter alia, composed of an accommodation 20 having a rail 21 and a clamping screw 22 and is adaptable on an objective of the microscope in such a manner that the objective is directly adjacent to an upper side 23 of connection device 14.

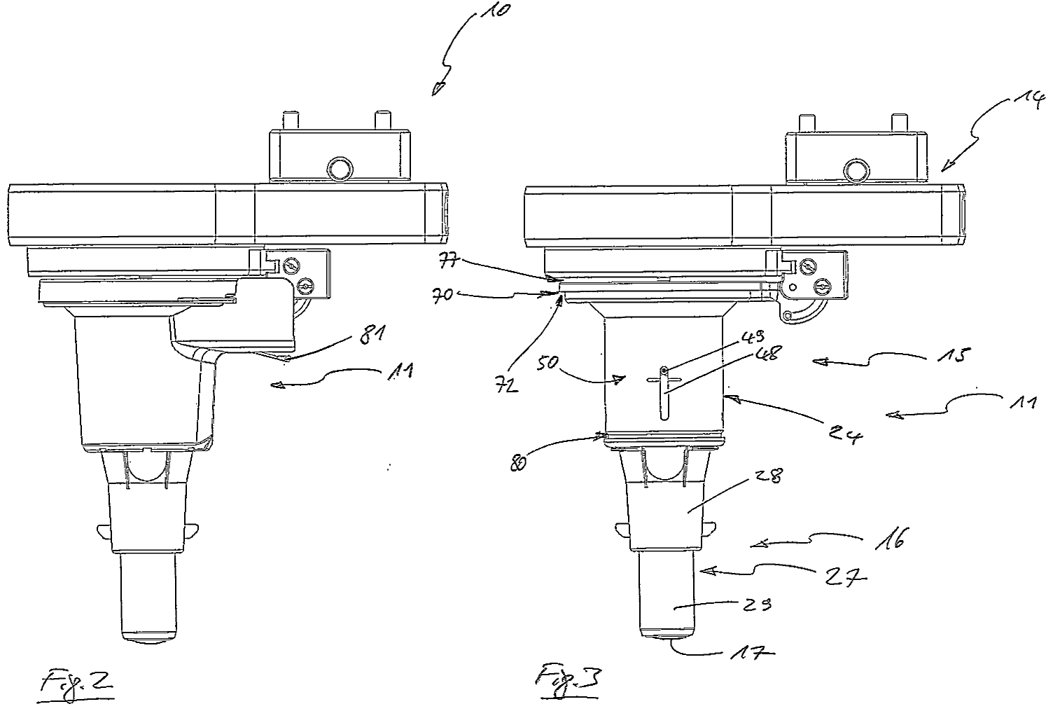

Accommodating device 16 is nearly completely made of plastic and holds ophthalmoscopy lens 17. Accommodating device 16 is adapted to positioning device 15. Positioning device 15 is made essentially of metal. By means of pivoting mechanism 19, which is realized by a hinge 25 in the case at hand, a lens barrel 24 of positioning device 15 can be pivoted by 90 degrees out of beam path 13 from an essentially vertical position in beam path 13 in such a manner that beam path 13 is cleared. Hinge 25 is realized with a guide 26 which allows a latching of positioning device 15 in the respective positions shown in FIGS. 2 and 4. Thus, positioning device 15 can be reliably fixed in the respective position.

Accommodating device 16 is realized by a further lens barrel 27 which is composed of an upper section 28 and of a lower section 29, in the case at hand. Furthermore, a compression spring 30 is inserted into upper section 28 and fixed by means of a ring 31 in upper section 28. Ophthalmoscopy lens 17 is held on a lower end 32 of accommodating device 16. Furthermore, lower section 29 is realized having two projections 33 each of which passes through a longitudinal slot 34 in upper section 28. Compression spring 30 is in contact with an upper edge 35 of lower section 29, lower section 29 resting on a step 37 on a lower end 38 of upper section 28 with an annular shoulder 36. In case of a collision of lower end 32 with an eye, lower section 29 can now be pushed into upper section 28 against a spring force of compression spring 30. Furthermore, it is also possible to grasp projections 33 with a hand and to push lower section 29 into upper section 28 in order to create a sufficient distance with respect to an eye when accommodating device 16 is to be pivoted together with positioning device 15.

Accommodating device 16 is realized with connecting elements 39 which engage into a groove 40 within lens barrel 27 and are latched there. Connecting elements 39 are realized on tabs 41 on an upper end 42 of upper section 28. Tabs 41 enable a springy mounting of connecting elements 39 transverse to beam path 13 and can be manually actuated. Pressing together and/or pronating tabs 41 then allows a removal of accommodating device 16 from lens barrel 24.

Lens barrel 24 is essentially composed of an outer sleeve 43 and an inner sleeve 44, inner sleeve 44 being rotatably mounted on a bearing 45 within lens barrel 24. A mount 46 with reduction lens 18 is inserted into inner sleeve 44. Furthermore, a helix 47 is realized in inner sleeve 44 and a slot 48 is realized in outer sleeve 43. Opposing Projections 49 on mount 46 each pass through helixes 47 and slots 48. A rotation of inner sleeve 44 relative to outer sleeve 43 causes such a movement of mount 46 with reduction lens 18 along beam path 13. A position of reduction lens 18 in lens barrel 28 is visible to a user on a circumferential surface 50 of lens barrel 24. In the case at hand, projections 49 are visible in slots 48.

When using observation device 10, ophthalmoscopy lens 17 can first be aligned with the eye by adjusting a height of the microscope. Subsequently, reduction lens 18 can be set by adjusting its position in lens barrel 24 in such a manner that an intermediate image of ophthalmoscopy lens 17 can be focused sharply by means of the microscope. The rotation of inner sleeve 44 in outer sleeve 43 is realized by a drive unit 51 of positioning device 15. In the case at hand, drive unit 51 is disposed on connection device 14 and comprises a step motor 52, a belt drive 53 and a coupling 54. In the case at hand, a drive wheel 55 of belt drive 53 is connected to an output sleeve 57 within connection device 14 via a belt 56. Output sleeve 57 surrounds beam path 13 coaxially and is rotatably mounted in a housing 59 of connection device 14 by means of a bearing 58. Coupling 54 is realized as a magnetic coupling 60, an axial end face 61 of inner sleeve 44 and an opposing axial end face 62 of output sleeve 57 each having magnets 63 embedded in them. Magnets 63 are disposed with alternating polarity in such a manner that opposing magnets 63 exert a magnetic force on each other so that a torque can be transferred to inner sleeve 44 when output sleeve 57 rotates.

A control device 64 of positioning unit 11 by means of which a rotation of lens barrel 24 and/or inner sleeve 44 can be controlled and detected is located within housing 59. Even if observation device 10 is turned or rotated on the microscope about beam path 13, for example with a manually executed rotation, it is thus possible to bring reduction lens 18 into a presupposed position in the longitudinal direction of beam path 13 by means of step motor 52 if reduction lens 18 has been adjusted and/or moved along beam path 13 by this rotation. For this purpose, control device 64 can be equipped with a sensor (not illustrated in the case at hand) for detecting the rotation. Furthermore, connections 65 for the connection to a power supply, a foot switch (not illustrated in the case at hand) and the microscope are provided on control device 64.

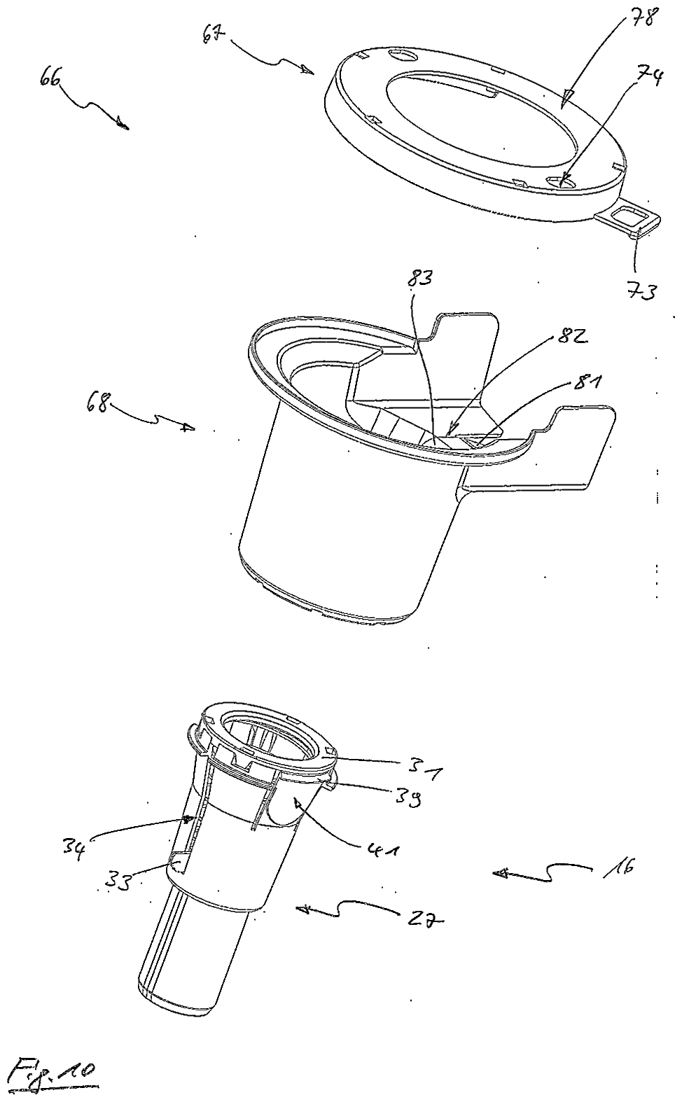

FIGS. 9 and 10 show accommodating device 16 together with a cover unit 66 of observation device 10. Cover unit 66 consists of a plastic material and is composed of an upper sterile cover 67 and a lower sterile cover 68. With upper sterile cover 67, an end surface 69 and an upper circumferential surface 70 of lens barrel 24 can be covered. With lower sterile cover 68, circumferential surface 50 of lens barrel 24 and partially hinge 25 can be covered. Upper sterile cover 67 has projections 71 which engage an upper annular shoulder 72 realized on lens barrel 24. Thus, upper sterile cover 67 can latch on upper annular shoulder 72. Furthermore, a tab 73 is provided on upper sterile cover 67 for manually removing upper sterile cover 67. Furthermore, recesses 74 are realized in upper sterile cover 67, pins 75 on connection device 14 passing through said recesses 74 in a mounting position. Pins 75 realize a stop 76 for lens barrel 24 and a gap 77 between lens barrel 24 and connection device 14, a circular covering area 78 of upper sterile cover 67 being accommodated within said gap 77 and being secured against turning by means of pins 75.

As upper sterile cover 67, lower sterile cover 68 is integrally formed and has projections 79 engaging into a lower annular groove 80 of lens barrel 24. Thus, lower sterile cover 68 can be fixed to a lens barrel 24 by latching. Furthermore, a tab 81 is realized on lower sterile cover 68, wherein lower sterile cover 68 can be removed from lens barrel 24 by means of said tab 81. Tab 81 is realized with lines of weakness 82 in lower cover 68 so that a tear strip 83 is formed, which leads to a destruction of lower sterile cover 68 when tab 81 is manually actuated. Thus, it can be guaranteed that cover unit 66 is not reused again after a removal. Furthermore, the plastic material of cover unit 66 is partially transparent.

Since cover unit 66 completely covers lens barrel 24, a sterilization of lens barrel 24 is not necessary after a surgery has been performed. After the surgery, cover unit 66 can be removed and replaced with a new sterile cover unit 66 which has not yet been used. This also applies to accommodating device 16 having ophthalmoscopy lens 17 so that an unintentional reuse and sterilization are in this case also precluded. Thus, after having performed a surgery, observation device 10 can be quickly prepared for a subsequent surgery by replacing cover unit 66 and accommodating device 16 without the need for a time-consuming sterilization of observation device 10.

1. An observation device (10) having a positioning unit (11) for positioning an optical unit (12) in a beam path (13) of a microscope between an objective of the microscope and before an eye to be observed, the positioning unit comprising a connection device (14), a positioning device (15), an accommodating device (16) and the optical unit, the optical unit comprising a lens, which serves to observe a fundus of the eye, and a further optical element, the positioning unit comprising a pivoting mechanism (19) by means of which the optical unit is pivotable out of or into the beam path, the positioning unit being couplable to the microscope by means of the connection device, the lens being adapted on the positioning device by means of the accommodating device, the accommodating device being at least predominantly, preferably completely, made of plastic material, wherein the positioning device has a lens barrel (24) which is disposed on the pivoting mechanism in a pivotable manner, the lens barrel being at least predominantly or completely made of metal.

2. The observation device according to claim 1, wherein the further optical element is disposed below the pivoting mechanism (19).

3. The observation device according to claim 1, wherein the further optical element is movable in the longitudinal direction of the beam path (13) relative to the microscope by means of the positioning device (15).

4. The observation device according to claim 1, wherein the lens is an ophthalmoscopy lens (17), the further optical element being at least one lens (18) having positive refractive power and serving to adjust the beam path, the lens in the lens barrel (24) being disposed movably in the longitudinal direction of the beam path below the pivoting mechanism (19).

5. The observation device according to claim 1, wherein the observation device (10) comprises a cover unit (66) made of plastic material for sterilely covering the pivotable lens barrel (24) of the positioning device (15).

6. The observation device according to claim 5, wherein the cover unit (66) has an upper sterile cover (67) for at least partially covering an end surface (69) of the lens barrel (24) and a lower sterile cover (68) for at least partially covering a circumferential surface (70) of the lens barrel.

7. The observation device according to claim 5, wherein the cover unit (66) is realized with at least one connecting element (71, 79) which requires a destruction of the connecting element when separating the cover unit from the positioning device (15).

8. The observation device according to claim 5, wherein the cover unit (66) has at least one tear strip (83) by means of which the cover unit is at least partially destroyable.

9. The observation device according to claim 5, wherein the accommodating device (16) is realized with at least one connecting element (39) which requires a destruction of the connecting element when separating the accommodating device from the positioning device (15).

10. The observation device according to claim 5, wherein the accommodating device (16) is realized having a further lens barrel (27).

11. The observation device according to claim 10, wherein the further lens barrel (27) is composed of an upper section (28) and of a lower section (29), the lower section being mounted loosely or in a spring-loaded manner on the upper section in such a manner that the lower section is insertable into the upper section.

12. The observation device according to claim 11, wherein at least one manually actuable projection (33) is realized on the lower section (29), the projection passing through a longitudinal slot (34), which is realized in the upper section (28), and being movable along the longitudinal slot.

13. The observation device according to claim 11, wherein the positioning device (15) has a drive unit (51) by means of which a position of the further optical element is settable in the longitudinal direction of the beam path (13).

14. The observation device according to claim 13, wherein the drive unit (51) is disposed on the connection device (14) above the pivoting mechanism (19) and/or below the pivoting mechanism on the lens barrel (24).

15. The observation device according to claim 11, wherein the observation device (10) comprises a shielding unit for shielding an optical path of the positioning unit (11), the shielding unit being composed of at least one optically shielded or closed lens barrel (24, 27).

16. A microscope having an observation device (10) according to claim 11.

17. A method for observing an eye with an observation device (10), an optical unit (12) being positioned in a beam path (13) of a microscope between an objective of the microscope and before an eye to be observed by means of a positioning unit (11) of the observation device, the positioning unit comprising a connection device (14), a positioning device (15), an accommodating device (16) and the optical unit, the optical unit comprising a lens, which serves to observe a fundus of the eye, and a further optical element, the positioning unit comprising a pivoting mechanism (19) by means of which the optical unit is pivoted out of or into the beam path, the positioning unit being coupled to the microscope by means of the connection device, the lens being adapted on the positioning device by means of the accommodating device, the accommodating device being at least predominantly made of plastic material, the method comprising pivoting a lens barrel (24) of the positioning device (15) is pivoted while disposed on the pivoting mechanism, the lens barrel being at least predominantly or completely made of metal.
